SEQUENCE LISTING (4 sequences)

SEQ ID NO: 1 - DNA, 31 bp, Artificial Sequence
  synthetic sequence
cgctcgagatgcaaagtgctatgttcttggc

SEQ ID NO: 2 - DNA, 32 bp, Artificial Sequence
  synthetic sequence
ccgctcgagatgcaaagtgctatgttcttggc

SEQ ID NO: 3 - DNA, 35 bp, Artificial Sequence
  synthetic sequence
ccgctcgagatgtgcaaagggcttgcaggtctgcc

SEQ ID NO: 4 - DNA, 29 bp, Artificial Sequence
  synthetic sequence
cgcggatccggcacactgagggaccaggg

(19) United States
(12) Patent Application Publication
(10) Pub. No.: US 2014/0336138 A1
(43) Pub. Date: Nov. 13, 2014

(54) COMPOSITIONS AND METHODS FOR SCREENING AND TREATMENT OF STURGE-WEBER SYNDROME, KLIPPEL-TRENAUNAY-WEBER SYNDROME, AND PORT-WINE STAINS (PWSS)

(71) Applicants: The Johns Hopkins University, Baltimore, MD (US); Kennedy Krieger Institute, Inc., Baltimore, MD (US)

(72) Inventors: Anne M. Comi, Baltimore, MD (US); Jonathan Pevsner, Baltimore, MD (US); Douglas Marchuk, Chapel Hill, NC (US)

(73) Assignees: The Johns Hopkins University, Baltimore, MD (US); Kennedy Krieger Institute, Inc., Baltimore, MD (US)

(21) Appl. No.: 14/271,739

(22) Filed: May 7, 2014

Related U.S. Application Data
(60) Provisional application No. 61/820,361, filed on May 7, 2013.

(51) Int. Cl. A61K 31/7048; C12Q 1/68; A61K 45/06; G01N 33/50

(52) U.S. Cl. CPC A61K 31/7048; G01N 33/5041; C12Q 1/6897; A61K 45/06; G01N 2500/02; C12Q 2600/136; C12Q 2600/158
USPC 514/26; 435/6.13; 506/10

(57) ABSTRACT

Compositions and methods for treatment of Sturge-Weber Syndrome, Klippel-Trenaunay-Weber Syndrome, Port-Wine Stains and related neurocutaneous disorders are provided. Cell lines having the somatic mutation GNAQ p.Arg183Gln amino acid substitution, which was found to be the cause of port-wine stains (prevalence 1 in 300) and Sturge-Weber syndrome are also provided. Compositions and methods for treatment of uveal melanoma are also provided herein. Methods of screening novel compounds and compositions useful in increasing RGS2 and/or RGS4 expression and function in vitro, and for treatment of Sturge-Weber Syndrome, Klippel-Trenaunay-Weber Syndrome, Port-Wine Stains and related neurocutaneous disorders are provided are also provided.

### REFERENCE TO RELATED APPLICATIONS

This application claims the benefit of U.S. Provisional Patent Application No. 61/820,361, filed on May 7, 2013, which is hereby incorporated by reference for all purposes as if fully set forth herein.

### INCORPORATION-BY-REFERENCE OF MATERIAL SUBMITTED ELECTRONICALLY

The instant application contains a Sequence Listing which has been submitted in ASCII format via EFS-Web and is hereby incorporated by reference in its entirety. Said ASCII copy, created on May 5, 2014, is named P12481-02_ST25.txt and is 1,123 bytes in size.

### STATEMENT OF GOVERNMENTAL INTEREST

This invention was made with government support under grant no. NS065705 awarded by the National Institutes of Health. The government has certain rights in the invention.

### BACKGROUND OF THE INVENTION

Sturge-Weber syndrome (SWS), also known as encephalofacial angiomatosis, is a neurocutaneous disorder that occurs as a sporadic, congenital condition characterized by a port-wine stain (PWS) affecting the V1 territory of the face (the forehead and/or eyelid) associated with a leptomengial angioma of the brain and venous abnormalities of the eye. It occurs in both males and females, in approximately 1 in 20-500,000 live births. Independently occurring port-wine stains are much more common, occurring in approximately 3 in 1000 births and commonly involve the head and neck. A child born with a port-wine stain on the face has approximately a 6% chance of having SWS, and this risk increases to 26% when the PWS is located in the V1 territory of the face. Port-wine stains commonly have underlying soft and bony tissue hypertrophy which may be mild or massive. When a port-wine stain-associated hypertrophy involves a limb and has enlarged venous or lymphatic vessels this is referred to as Klippel-Trenaunay Weber syndrome (KTWS). KTWS has been reported in association with SWS when the PWS is extensive and extends down on to the trunk and effected limb.

Guanine nucleotide binding protein, q polypeptide (GNAQ) encodes a guanine nucleotide associated protein (q polypeptide; Gα_{q}) that forms the a subunit of a heterotrimeric Gq protein complex essential to the intracellular signaling of a subset of G protein coupled receptors (GPCRs). When the GPCR ligand binds to the inactive receptor this induces a conformational change in the GPCR and the activated receptor binds to Gα_{q}. In the inactive state, Gα_{q} binds GDP; when Gα_{q} is bound to the activated receptor GDP is released and GTP is bound instead. Binding of GTP to Gα_{q} triggers dissociation of Gα_{q} from both the receptor and the Gβ subunit of the trimeric Gq protein. Gα_{q} then binds to one of its effectors (such as phospholipase C-β or Trio, for example) thereby activating the effector. Gα_{q} is capable of auto-hydrolysis of GTP to GDP at its binding site, causing Gα_{q} to dissociate from its effector, reassociate with the GP subunit, and return to the G protein to its inactive trimeric state.

The somatic mutation GNAQ p.Arg183Gln amino acid substitution was recently found by the inventors to be the cause of PWS, SWS and KTWS (see, U.S. Provisional Application No. 61/812,309, and incorporated by reference in its entirety herein). The mutation results in a cysteine substitution at Gα_{q}Arg183, which is conserved in the GTP binding pocket of all 24 human Gα subunits. Substitution of cysteine at this position causes impaired auto-hydrolysis of GTP to GDP, thus resulting in relative inability of Gα_{q} to cease effector stimulation and constitutive activation of the downstream pathways. Confirmation of this was found in increased phosphorylated-ERK and phosphorylated-JNK in cells transfected with mutated constructs, when compared to wild-type. Importantly, the p.Arg183Gln mutation in GNAQ does not result in as severe dysregulation as does the p.Gln209Leu mutation in GNAQ which is associated with uveal melanoma; the degree of constitutive hyperactivation of downstream effectors (such as ERK) is significantly less.

Many treatments have been tried for port-wine stains including freezing, surgery, radiation, and tattooing; port-wine stains can also be covered with cosmetics. Lasers have made the biggest impact on treatment, because they are the sole method of destroying the cutaneous capillaries without significant damage to the overlying skin. However, complete disappearance is rare. In approximately 20% of cases, there may be no improvement at all. Stains on the face respond better than those on the trunk or limbs. Older stains may be more difficult to treat. In the absence of successful treatment, hypertrophy (increased tissue mass) of the stains may produce deformity, loss of function (especially near the eye or mouth), bleeding, and increasing disfigurement. These complications are usually seen later in life. If the port-wine stain is on the face or other highly visible part of the body, its presence can also cause emotional and social problems for the affected person.

Therefore, there still exists a need to develop significantly improved therapies for treatment of PWS, SWS and KTWS.

### SUMMARY OF THE INVENTION

In accordance with one or more embodiments, the present inventors have now identified that one or more classes of compounds can be useful in the treatment of PWS, SWS and KTWS. Further, with knowledge of the somatic mutation which was found by the present inventors to be the cause of PWS, SWS and KTWS, methods for screening molecules which are useful for the treatment of PWS, SWS and KTWS are disclosed herein.

A group of proteins are known to regulate the G protein/GPCR cycle described above; these are the Regulator of G Protein Signaling (RGS) proteins. Normally, RGS proteins enhance hydrolysis of the Gα subunit, thereby modulating inactivation of both the effector and trimeric G protein.

Regulator of G protein signaling 2 (RGS2), a Gq-specific GTPase activator protein (GAP), is strongly implicated in cardiovascular function. RGS2−/− mice are hypertensive and prone to heart failure and several rare human mutations that speed RGS2 degradation have been identified in hypertensive patients. While it is also known that RGS4 can also stimulate the Gq-specific GTPAase in a non-specific manner, it is thought that elevation of RGS4 function may also be useful in the methods of the present invention.

Several cardiotonic steroids (CTS), including ouabain and digoxin, increase RGS2 but not RGS4 protein levels. CTS increase RGS2 protein levels through a posttranscriptional mechanism by slowing protein degradation. RGS2 mRNA levels in primary vascular smooth muscle cells are unaffected by CTS treatment while protein levels are increased 2-3 fold. Moreover, CTS-induced increases in RGS2 are functional, reducing receptor-stimulated Gq-dependent ERK phosphorylation. Thus, the present inventors hypothesized that administration of CTS could be used to treat PWS, SWS and KTWS by increasing endogenous RGS2 in the cells, and correcting the constitutive hyperphosphorylation of ERK and JNK was already discovered in these cells by the inventors.

In accordance with an embodiment, the present invention provides a method for treating Sturge-Weber Syndrome, Klippel-Trenaunay-Weber Syndrome, Port-Wine Stains and related neurocutaneous disorders in a subject comprising administering to the subject a therapeutically effective amount of a pharmaceutically acceptable composition which increases RGS2 and/or RGS4 protein levels in the subject.

In accordance with another embodiment, the present invention provides a method for treatment of a GNAQ dependent melanoma in a subject comprising administering to the subject a therapeutically effective amount of a pharmaceutically acceptable composition which increases RGS2 and/or RGS4 protein levels in the subject.

In accordance with further embodiments, the pharmaceutically acceptable composition which increases RGS2 and/or RGS4 protein levels is a composition comprising at least one cardiotonic steroid. In another embodiment, the pharmaceutically acceptable composition which increases RGS2 and/or RGS4 protein levels is a composition comprising at least one cardiotonic steroid and at least one other biologically active agent, such as a chemotherapeutic agent.

In accordance with another embodiment, the present invention provides a method for identifying a molecule which increases RGS2 and/or RGS4 protein levels in a cell or population of cells having the GNAQ Arg183Gln amino acid substitution comprising: a) obtaining a cell or population of cells which express GNAQ having the Arg183Gln amino acid substitution and a cell or population of cells having wild type GNAQ; b) incubating the molecule with both cells or population of cells of a); c) measuring the levels of RGS2 and or RGS4, in both cells or population of cells of a); d) comparing the levels of RGS2 and or RGS4 in both cells or population of cells of a); and e) determining that the molecule increases RGS2 and/or RGS4 protein levels in a cell or population of cells having the GNAQ Arg183Gln amino acid substitution when the protein levels are greater than in the cell or population of cells having wild type GNAQ.

In accordance with a further embodiment, the present invention provides A method for identifying a molecule which increases RGS2 and/or RGS4 protein levels in a cell or population of cells having the GNAQ Arg183Gln amino acid substitution comprising: a) obtaining a cell or population of cells which express RGS2 and/or RGS4 protein which are capable of emitting a photon when stimulated; b) incubating the molecule with the cell or population of cells of a); c) measuring the levels of photo emission, in the cell or population of cells of a); d) comparing the levels of RGS2 and or RGS4 in the cell or population of cells of a) to that of a control; and e) determining that the molecule increases RGS2 and/or RGS4 protein levels in a cell or population of cells when the protein levels are greater than the control.

### BRIEF DESCRIPTION OF THE DRAWINGS

FIG. 1 is a depiction of the chemical structures of some well-known cardiotonic steroids.

FIG. 2 is data from U.S. Provisional Application No. 61/812,309, showing transiently transfected 293T cells with mutant GNAQ constructs have been shown to demonstrate constitutive hyperphosphorylation of ERK and JNK. Plasmid encoding non-mutant Gαq, p.Arg183Gln, and p.Gln209Leu were transfected into human embryonic kidney (HEK) 293T cells. Strongly increased phosphorylation of extracellular signal-regulated kinase (ERK) is seen with Gαq p.Gln209Leu, and weaker but marked activation with Gαq p.Arg183Gln (P<0.05 for both comparisons) (Panel A). Increased phosphorylation of p38 is seen with Gαq p.Gln209Leu (P<0.05) but not with Gαq p.Arg183Gln (Panel B). Increased phosphorylation of Jun N-terminal kinase (JNK) is seen with Gαq p.Gln209Leu (P<0.05), and weaker activation with Gαq p.Arg183Gln (P=0.052) (Panel C). No change in phosphorylation of AKT is seen with either the Gαq p.Arg183Gln or the p.Gln209Leu construct (Panel D). A control for transfection efficiency, transfected into HEK 293T cells, shows similar amounts of the three transfected, Flag-tagged proteins (Panel E). A serum response-element (SRE) luciferase assay (Panel F) shows the relative luciferase activity expressed under the control of the SRE promoter, co-expressed with GNAQ encoding p.Arg183Gln and p.Gln209Leu, as compared with non-mutant Gαq (P<0.05 for both comparisons). AU denotes arbitrary units, the prefix p antibody recognizing phosphorylated antigen, and the prefix t antibody recognizing total antigen.

### DETAILED DESCRIPTION OF THE INVENTION

As used herein, the term “GNAQ” refers to the alpha subunit of a guanine nucleotide binding protein (G-protein). The term encompasses nucleic acid and polypeptide polymorphic variants, alleles, mutants, and fragments of GNAQ. Such sequences are well known in the art. Exemplary human Gnaq sequences are available under the reference sequences NM_{—}002072 in the NCBI nucleotide database (nucleotide sequence) (and NM_{—}002072.3) and accession number NP_{—}002063.2 (polypeptide sequence).

A “GNAQ-dependent mutation” as used in the context of this application refers to cells that have a defect in GNAQ that activates or otherwise disrupts the function of GNAQ, i.e., has an “activating” mutation, in comparison to cells that do not have the mutation, and leads to a loss or decrease of GTP hydrolyzing activity of the mutant G-α subunit. The GNAQ mutation, e.g., a substitution mutation, can result in constitutive activity of the protein. The “GNAQ-dependent mutation” may have one or more of such mutations, e.g., the cells may have somatic substitution mutation involving R183. A “GNAQ-dependent mutation” may also have mutations in genes other than GNAQ.

The terms “sample,” “patient sample,” “biological sample,” and the like, encompass a variety of sample types obtained from a patient, individual, or subject and can be used in a diagnostic, prognostic or monitoring assay. The patient sample may be obtained from a healthy subject, a diseased patient including, for example, a patient having associated symptoms of SWS, KTWS or PWS. Moreover, a sample obtained from a patient can be divided and only a portion may be used for diagnosis, prognosis or monitoring. Further, the sample, or a portion thereof, can be stored under conditions to maintain sample for later analysis. The definition specifically encompasses blood and other liquid samples of biological origin (including, but not limited to, peripheral blood, serum, plasma, urine, saliva, amniotic fluid, stool and synovial fluid), solid tissue samples such as a biopsy specimen or tissue cultures or cells derived therefrom and the progeny thereof. In a specific embodiment, a sample comprises a skin sample. In another embodiment, a sample of brain tissue is used. In other embodiments, a sample comprises a blood or serum sample. The definition also includes samples that have been manipulated in any way after their procurement, such as by centrifugation, filtration, precipitation, dialysis, chromatography, treatment with reagents, washed, or enriched for certain cell populations. The terms further encompass a clinical sample, and also include cells in culture, cell supernatants, tissue samples, organs, and the like. Samples may also comprise fresh-frozen and/or formalin-fixed, paraffin-embedded tissue blocks, such as blocks prepared from clinical or pathological biopsies, prepared for pathological analysis or study by immunohistochemistry.

The terms “providing a sample” and “providing a biological (or patient) sample” are used interchangeably and mean to provide or obtain a biological sample for use in methods described in this invention. Most often, this will be done by removing a sample of cells from a patient, but can also be accomplished by using previously isolated cells (e.g., isolated by another person, at another time, and/or for another purpose), or by performing the methods of the invention in vivo. Archival tissues, having treatment or outcome history, can also be used.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, .gamma.-carboxyglutamate, and O-phosphoserine. “Amino acid analogs” refers to compounds that have the same basic chemical structure as a naturally occurring amino acid, e.g., an .alpha. carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As to amino acid sequences, one of ordinary skill in the art recognizes that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. Typical conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

“Nucleic acid” or “oligonucleotide” or “polynucleotide” or grammatical equivalents used herein means at least two nucleotides covalently linked together. Oligonucleotides are typically from about 5, 6, 7, 8, 9, 10, 12, 15, 25, 30, 40, 50 or more nucleotides in length, up to about 100 nucleotides in length. Nucleic acids and polynucleotides are polymers of any length, including longer lengths, e.g., 200, 300, 500, 1000, 2000, 3000, 5000, 7000, 10,000, etc. A nucleic acid of the present invention will generally contain phosphodiester bonds, although in some cases, nucleic acid analogs are included that may have alternate backbones, comprising, e.g., phosphoramidate, phosphorothioate, phosphorodithioate, or O-methylphophoroamidite linkages (see Eckstein, Oligonucleotides and Analogues: A Practical Approach, Oxford University Press); and peptide nucleic acid backbones and linkages. Other analog nucleic acids include those with positive backbones; non-ionic backbones, and non-ribose backbones, including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made.

The nucleic acids may be single stranded or double stranded, as specified, or contain portions of both double stranded or single stranded sequence. As will be appreciated by those in the art, the depiction of a single strand also defines the sequence of the complementary strand; thus the sequences described herein also provide the complement of the sequence. Unless otherwise indicated, a particular nucleic acid sequence also implicitly encompasses conservatively modified variants thereof (e.g., degenerate codon substitutions) and complementary sequences, as well as the sequence explicitly indicated. The nucleic acid may be DNA, both genomic and cDNA, RNA or a hybrid, where the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, etc. “Transcript” typically refers to a naturally occurring RNA, e.g., a pre-mRNA, hnRNA, or mRNA. As used herein, the term “nucleoside” includes nucleotides and nucleoside and nucleotide analogs, and modified nucleosides such as amino modified nucleosides. In addition, “nucleoside” includes non-naturally occurring analog structures. Thus, e.g. the individual units of a peptide nucleic acid, each containing a base, are referred to herein as a nucleoside.

A “label” or a “detectable moiety” is a composition detectable by spectroscopic, photochemical, biochemical, immunochemical, chemical, or other physical means. For example, useful labels include ³²P, fluorescent dyes, near infra-red dyes, electron-dense reagents, enzymes (e.g., as commonly used in an ELISA), biotin, digoxigenin, or haptens and proteins or other entities which can be made detectable, e.g., by incorporating a radiolabel into the peptide or used to detect antibodies specifically reactive with the peptide. The labels may be incorporated into the KIT nucleic acids, proteins and antibodies at any position. Any method known in the art for conjugating the antibody to the label may be employed, e.g., using methods described in Hermanson, Bioconjugate Techniques 1996, Academic Press, Inc., San Diego.

A “labeled nucleic acid probe or oligonucleotide” is one that is bound, either covalently, through a linker or a chemical bond, or noncovalently, through ionic, van der Waals, electrostatic, or hydrogen bonds to a label such that the presence of the probe may be detected by detecting the presence of the label bound to the probe. Alternatively, method using high affinity interactions may achieve the same results where one of a pair of binding partners binds to the other, e.g., biotin, streptavidin.

As used herein a “nucleic acid probe or oligonucleotide” is defined as a nucleic acid capable of binding to a target nucleic acid of complementary sequence through one or more types of chemical bonds, usually through complementary base pairing, usually through hydrogen bond formation. As used herein, a probe may include natural (i.e., A, G, C, or T) or modified bases (7-deazaguanosine, inosine, etc.). In addition, the bases in a probe may be joined by a linkage other than a phosphodiester bond, so long as it does not functionally interfere with hybridization. Thus, e.g., probes may be peptide nucleic acids in which the constituent bases are joined by peptide bonds rather than phosphodiester linkages. It will be understood by one of skill in the art that probes may bind target sequences lacking complete complementarity with the probe sequence depending upon the stringency of the hybridization conditions. The probes are preferably directly labeled as with isotopes, chromophores, lumiphores, chromogens, or indirectly labeled such as with biotin to which a streptavidin complex may later bind. By assaying for the presence or absence of the probe, one can detect the presence or absence of the select sequence or subsequence.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, e.g., recombinant cells express genes that are not found within the native (non-recombinant) form of the cell or express native genes that are otherwise abnormally expressed, under expressed or not expressed at all. By the term “recombinant nucleic acid” herein is meant nucleic acid, originally formed in vitro, in general, by the manipulation of nucleic acid, e.g., using polymerases and endonucleases, in a form not normally found in nature. Similarly, a “recombinant protein” is a protein made using recombinant techniques, i.e., through the expression of a recombinant nucleic acid as depicted above.

As used herein, “antibody” includes reference to an immunoglobulin molecule immunologically reactive with a particular antigen, and includes both polyclonal and monoclonal antibodies. The term also includes genetically engineered forms such as chimeric antibodies (e.g., humanized murine antibodies) and heteroconjugate antibodies (e.g., bispecific antibodies). The term “antibody” also includes antigen binding forms of antibodies, including fragments with antigen-binding capability (e.g., Fab′, F(ab′).sub.2, Fab, Fv and rIgG. See also, Pierce Catalog and Handbook, 1994-1995 (Pierce Chemical Co., Rockford, Ill.). See also, e.g., Kuby, J., Immunology, 3.sup.rd Ed., W.H. Freeman & Co., New York (1998). The term also refers to recombinant single chain Fv fragments (scFv). The term antibody also includes bivalent or bispecific molecules, diabodies, triabodies, and tetrabodies. Bivalent and bispecific molecules are described in, e.g., Kostelny et al. (1992) J Immunol 148:1547, Pack and Pluckthun (1992) Biochemistry 31:1579, Hollinger et al., 1993, supra, Gruber et al. (1994) J Immunol: 5368, Zhu et al. (1997) Protein Sci 6:781, Hu et al. (1996) Cancer Res. 56:3055, Adams et al. (1993) Cancer Res. 53:4026, and McCartney, et al. (1995) Protein Eng. 8:301.

The phrase “functional effects” in the context of assays for testing compounds that inhibit activity of a GNAQ protein includes the determination of a parameter that is indirectly or directly under the influence of the GNAQ protein or nucleic acid, e.g., a functional, physical, or chemical effect, such as the ability to alter GTP hydrolase activity. Activities or functional effect of GNAQ can include protein-protein interaction activity, e.g., the ability of GNAQ to bind an antibody or other protein with which it interacts; GTP hydrolase activity, the ability of GNAQ to bind GTP and/or GDP; contact inhibition and density limitation of growth; cellular proliferation; cellular transformation; changes in pigmentation; growth factor or serum dependence; and mRNA and protein expression in cells. “Functional effects” include in vitro, in vivo, and ex vivo activities.

As used herein, “inhibitors” or “antagonists” of RGS2 and/or RGS4 (e.g. “RGS antagonists”) refer to modulatory molecules or compounds that, e.g., bind to, partially or totally block activity, decrease, prevent, delay activation, inactivate, desensitize, or down regulate the activity or expression of RGS2/4 protein.

As used herein, the terms “stimulators” or “agonists of RGS2 and/or RGS4 (e.g. “RGS agonists”), refer to modulatory molecules or compounds that, e.g., bind to, partially or totally stimulate activity, increase, promote activation, activate, sensitize, or up regulate the activity or expression of RGS2/4 protein.

In accordance with one or more embodiments, stimulators of RGS2 and/or RGS4 include, for example, cardiotonic steroids or cardiac glycosides. The cardiac glycosides are an important class of naturally occurring drugs whose actions include both beneficial and toxic effects on the heart. Plants containing cardiac steroids have been used as poisons and heart drugs at least since 1500 B.C. Throughout history these plants or their extracts have been variously used as arrow poisons, emetics, diuretics, and heart tonics. Cardiac steroids are widely used in the modern treatment of congestive heart failure and for treatment of atrial fibrillation and flutter. The R group at the 17-position defines the class of cardiac glycoside. Two classes have been observed in nature, the cardenolides, and the bufadienolides. The cardenolides have an unsaturated butyrolactone ring while the bufadienolides have an a-pyrone ring. The steroid nucleus has hydroxyls at 3- and 14-positions of which the sugar attachment uses the 3-OH group. 14-OH is normally unsubstituted. Many genins have OH groups at 12- and 16-positions. These additional hydroxyl groups influence the partitioning of the cardiac glycosides into the aqueous media and greatly affect the duration of action. The lactone moiety at C-17 position is an important structural feature. The size and degree of unsaturation varies with the source of the glycoside. Normally plant sources provide a 5-membered unsaturated lactone while animal sources give a 6-membered unsaturated lactone.

One to four sugars are found to be present in most cardiac glycosides attached to the 3b-OH group. The sugars most commonly used include L-rhamnose, D-glucose, D-digitoxose, D-digitalose, D-digginose, D-sarmentose, L-vallarose, and D-fructose. These sugars predominantly exist in the cardiac glycosides in the b-conformation. The presence of acetyl group on the sugar affects the lipophilic character and the kinetics of the entire glycoside. Because the order of sugars appears to have little to do with biological activity Nature has synthesized a repertoire of numerous cardiac glycosides with differing sugar skeleton but relatively few aglycone structures.

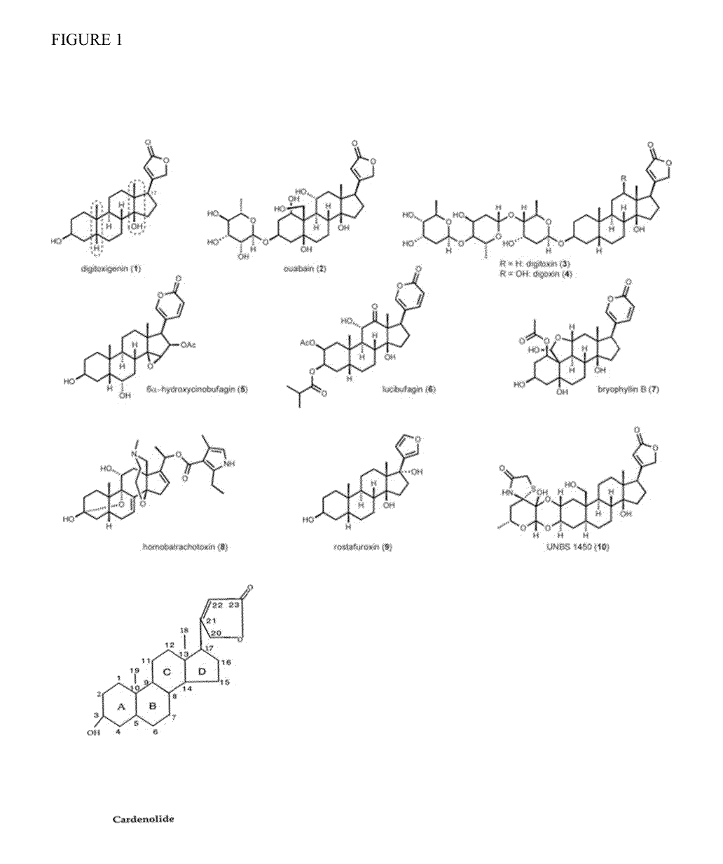

Some examples of cardiac steroids useful in accordance with the methods of the present invention include, but are not limited to, ouabain, deslanoside, digoxin, digitoxin, lanatoside C, acetyldigoxin, cardenolide, and G-strophanthin (FIG. 1).

In some embodiments, assays comprising cells that express RGS2/4 proteins that are treated with a potential stimulator are compared to control samples without the stimulator, to examine the effect on activity. Typically, control samples, e.g., cells that express RGS2/4 proteins and that are untreated with stimulators are assigned a relative protein activity value of 100%. Stimulation of RGS2/4 protein levels is achieved when the activity value relative to the control is changed at least about 20%, at least about 50%, at least about 75-100%, or more.

In accordance with one or more embodiments, assays comprising cells that express GNAQ Arg183Gln amino acid substitution and which express RGS2/4 proteins are treated with a potential stimulator are compared to control samples without the stimulator, to examine the effect on activity. Typically, control samples, e.g., cells that express RGS2/4 proteins and that are untreated with stimulators are assigned a relative protein activity value of 100%. Stimulation of RGS2/4 protein levels is achieved when the activity value relative to the control is changed at least about 20%, at least about 50%, at least about 75-100%, or more.

The compounds tested as stimulators of RGS2/4 protein levels and expression can be any small chemical compound, or a biological entity, e.g., a macromolecule such as a protein, sugar, nucleic acid or lipid. Typically, test compounds will be small chemical molecules and peptides or antibodies.

In some embodiments, the agents have a molecular weight of less than 1,500 Daltons, and in some cases less than 1,000, 800, 600, 500, or 400 Daltons. The relatively small size of the agents can be desirable because smaller molecules have a higher likelihood of having physiochemical properties compatible with good pharmacokinetic characteristics, including oral absorption than agents with higher molecular weight. For example, agents less likely to be successful as drugs based on permeability and solubility were described by Lipinski et al. as follows: having more than 5 H-bond donors (expressed as the sum of OHs and NHs); having a molecular weight over 500; having a Log P over 5 (or M Log P over 4.15); and/or having more than 10 H-bond acceptors (expressed as the sum of Ns and Os). See, e.g., Lipinski et al. Adv Drug Delivery Res 23:3-25 (1997). Compound classes that are substrates for biological transporters are typically exceptions to the rule.

Essentially any chemical compound can be used as a potential stimulator or ligand in the assays of the present invention. Most often, compounds can be dissolved in aqueous or organic (especially DMSO-based) solutions. The assays are designed to screen large chemical libraries by automating the assay steps, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

Expression of RGS2/4 levels can be detected in a number of different ways. As described herein, the expression levels of the protein in a cell can be determined by probing the mRNA expressed in a cell with a probe that specifically hybridizes with a RGS2/4 transcript (or complementary nucleic acid derived therefrom). Alternatively, protein can be detected using immunological methods in which a cell lysate is probed with antibodies that specifically bind to the protein.

Other cell-based assays are reporter assays can be conducted with cells that do not express the protein. Often, these assays are conducted with a heterologous nucleic acid construct that includes a promoter that is operably linked to a reporter gene that encodes a detectable product.

Uveal melanoma is a melanocytic neoplasm that arises from melanocytes in the choroidal plexus, ciliary body or iris epithelium of the eye (e.g., Singh, et al., Ophthalmol Clin North Am 18:75-84, viii, 2005). In more aggressive subtypes there are further genetic alterations such as monosomy 3, trisomy 8 and a strong tendency to metastasize to the liver (Singh, et al., Ophthalmol Clin North Am 18:75-84, viii, 2005; Horsman & White, Cancer 71:811-9, 1993). Uveal melanoma is highly aggressive, with a 5-year disease-specific survival rate of approximately 70% (e.g., Chang et al., Cancer 83:1664-78, 1998). One risk factor for uveal melanoma is the presence of bluish-grey hyper-pigmentation in the conjunctiva and periorbital dermis, called the naevus of Ota (Singh et al., Ophthalmology 105:195-8, 1998). (1998) Am J Dermatopathol. 20:109-110).

It has recently been found that activating mutations in exon 4 of GNA11 and GNAQ occur in melanocytic neoplasms, including in uveal melanoma (GNAQ dependent melanoma). The mutation is an exon 4 mutation in GNAQ or GNA11 mutation, e.g., at R183 or V182, which is the same substitution shown to be responsible for PWS and related diseases by the present inventors (See, U.S. Patent Publication No, 2013/0102653).

Thus, in accordance with an embodiment, the present invention provides a method for treatment of a GNAQ dependent melanoma in a subject comprising administering to the subject a therapeutically effective amount of a pharmaceutically acceptable composition which increases RGS2 and/or RGS4 protein levels in the subject.

In some embodiments, the pharmaceutically acceptable composition which increases RGS2 and/or RGS4 protein levels is a composition comprising at least one cardiotonic steroid. In another embodiment, the pharmaceutically acceptable composition which increases RGS2 and/or RGS4 protein levels is a composition comprising at least one cardiotonic steroid and at least one other biologically active agent, such as a chemotherapeutic agent. Examples of chemotherapeutic agents include antineoplastic agents, such as alkylating agents, nitrogen mustard alkylating agents, nitrosourea alkylating agents, antimetabolites, purine analog antimetabolites, pyrimidine analog antimetabolites, hormonal antineoplastics, natural antineoplastics, antibiotic natural antineoplastics, and vinca alkaloid natural antineoplastics.

“Treating” or “treatment” is an art-recognized term which includes curing as well as ameliorating at least one symptom of any condition or disease. Treating includes reducing the likelihood of a disease, disorder or condition from occurring in an animal which may be predisposed to the disease, disorder and/or condition but has not yet been diagnosed as having it; inhibiting the disease, disorder or condition, e.g., impeding its progress; and relieving the disease, disorder or condition, e.g., causing any level of regression of the disease; inhibiting the disease, disorder or condition, e.g., impeding its progress; and relieving the disease, disorder or condition, even if the underlying pathophysiology is not affected or other symptoms remain at the same level.

“Prophylactic” or “therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if it is administered after manifestation of the unwanted condition, the treatment is therapeutic (i.e., it is intended to diminish, ameliorate, or stabilize the existing unwanted condition or side effects thereof).

The compositions of the present invention may include a carrier. The term, “carrier,” refers to a diluent, adjuvant, excipient or vehicle with which the therapeutic is administered. Such physiological carriers can be sterile liquids, such as water and oils, including those of petroleum, animal, vegetable or synthetic origin, such as peanut oil, soybean oil, mineral oil, sesame oil and the like. Water is a suitable carrier when the pharmaceutical composition is administered intravenously. Saline solutions and aqueous dextrose and glycerol solutions also can be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents.

The compositions of the present invention may include a surfactant. As used herein, the term “surfactant” refers to organic substances having amphipathic structures, namely, are composed of groups of opposing solubility tendencies, typically an oil-soluble hydrocarbon chain and a water-soluble ionic group. Surfactants can be classified, depending on the charge of the surface-active moiety, into anionic, cationic and nonionic surfactants. Surfactants often are used as wetting, emulsifying, solubilizing and dispersing agents for various pharmaceutical compositions and preparations of biological materials.

The compositions of the present invention may include an active agent. An active agent and a biologically active agent are used interchangeably herein to refer to a chemical or biological compound that induces a desired pharmacological and/or physiological effect, wherein the effect may be prophylactic or therapeutic. The terms also encompass pharmaceutically acceptable, pharmacologically active derivatives of those active agents specifically mentioned herein, including, but not limited to, salts, esters, amides, prodrugs, active metabolites, analogs and the like. When the terms “active agent,” “pharmacologically active agent” and “drug” are used, then, it is to be understood that the invention includes the active agent per se as well as pharmaceutically acceptable, pharmacologically active salts, esters, amides, prodrugs, metabolites, analogs etc. The active agent can be a biological entity, such as a virus or cell, whether naturally occurring or manipulated, such as transformed.

The compositions of the present invention may include pharmaceutically acceptable salts. Pharmaceutically acceptable salts are art-recognized, and include relatively non-toxic, inorganic and organic acid addition salts of compositions of the present invention, including without limitation, therapeutic agents, excipients, other materials and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc and the like. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di-, or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; N-benzylphenthylamine; (trihydroxymethyl)aminoethane; and the like, see, for example, J. Pharm. Sci., 66: 1-19 (1977).

In one aspect of this invention, a composition comprising a RGS2 and/or RGS4 stimulator and one or more biologically active agents may be prepared. The biologically active agent may vary widely with the intended purpose for the composition. The term active is art-recognized and refers to any moiety that is a biologically, physiologically, or pharmacologically active substance that acts locally or systemically in a subject. Examples of biologically active agents, that may be referred to as “drugs”, are described in well-known literature references such as the Merck Index, the Physicians' Desk Reference, and The Pharmacological Basis of Therapeutics, and they include, without limitation, medicaments; vitamins; mineral supplements; substances used for the treatment, prevention, diagnosis, cure or mitigation of a disease or illness; substances which affect the structure or function of the body; or pro-drugs, which become biologically active or more active after they have been placed in a physiological environment. Various forms of a biologically active agent may be used which are capable of being released the subject composition, for example, into adjacent tissues or fluids upon administration to a subject.

Further examples of biologically active agents include, without limitation, enzymes, receptor antagonists or agonists, hormones, growth factors, autogenous bone marrow, antibiotics, antimicrobial agents, and antibodies.

In certain embodiments, the subject compositions of the present invention comprise about 1% to about 75% or more by weight of the total composition, alternatively about 2.5%, 5%, 10%, 20%, 30%, 40%, 50%, 60% or 70%, of a biologically active agent.

Various forms of the biologically active agents may be used. These include, without limitation, such forms as uncharged molecules, molecular complexes, salts, ethers, esters, amides, prodrug forms and the like, which are biologically activated when implanted, injected or otherwise placed into a subject.

In certain embodiments of the present invention, other materials may be incorporated into subject compositions in addition to one or more biologically active agents. For example, plasticizers and stabilizing agents known in the art may be incorporated in compositions of the present invention. In certain embodiments, additives such as plasticizers and stabilizing agents are selected for their biocompatibility or for the resulting physical properties of the reagents.

Buffers, acids and bases may be incorporated in the compositions of the present invention to adjust pH. Agents to increase the diffusion distance of agents released from the composition may also be included.

The charge, lipophilicity or hydrophilicity of a composition of the present invention may be modified by employing an additive. For example, surfactants may be used to enhance miscibility of poorly miscible liquids. Examples of suitable surfactants include dextran, polysorbates and sodium lauryl sulfate. In general, surfactants are used in low concentrations, generally less than about 5%.

The specific method used to formulate the novel formulations of the present invention described herein is not critical to the present invention and can be selected from a physiological buffer (Feigner et al., U.S. Pat. No. 5,589,466 (1996)).

Therapeutic formulations of the compositions of the present invention may be prepared for storage as lyophilized formulations or aqueous solutions by mixing the product having the desired degree of purity with optional pharmaceutically acceptable carriers, diluents, excipients or stabilizers typically employed in the art, i.e., buffering agents, stabilizing agents, preservatives, isotonifiers, non-ionic detergents, antioxidants and other miscellaneous additives, see Remington's Pharmaceutical Sciences, 16th ed., Osol, ed. (1980). Such additives are generally nontoxic to the recipients at the dosages and concentrations employed, hence, the excipients, diluents, carriers and so on are pharmaceutically acceptable.

The compositions of the present invention can take the form of solutions, suspensions, emulsions, powders, sustained-release formulations, depots and the like. Examples of suitable carriers are described in “Remington's Pharmaceutical Sciences,” Martin. Such compositions will contain an effective amount of the biopolymer of interest, preferably in purified form, together with a suitable amount of carrier so as to provide the form for proper administration to the patient. As known in the art, the formulation will be constructed to suit the mode of administration.

Buffering agents help to maintain the pH in the range which approximates physiological conditions. Buffers are preferably present at a concentration ranging from about 2 mM to about 50 mM. Suitable buffering agents for use with the instant invention include both organic and inorganic acids, and salts thereof, such as citrate buffers (e.g., monosodium citrate-disodium citrate mixture, citric acid-trisodium citrate mixture, citric acid-monosodium citrate mixture etc.), succinate buffers (e.g., succinic acid monosodium succinate mixture, succinic acid-sodium hydroxide mixture, succinic acid-disodium succinate mixture etc.), tartrate buffers (e.g., tartaric acid-sodium tartrate mixture, tartaric acid-potassium tartrate mixture, tartaric acid-sodium hydroxide mixture etc.), fumarate buffers (e.g., fumaric acid-monosodium fumarate mixture, fumaric acid-disodium fumarate mixture, monosodium fumarate-disodium fumarate mixture etc.), gluconate buffers (e.g., gluconic acid-sodium glyconate mixture, gluconic acid-sodium hydroxide mixture, gluconic acid-potassium gluconate mixture etc.), oxalate buffers (e.g., oxalic acid-sodium oxalate mixture, oxalic acid-sodium hydroxide mixture, oxalic acid-potassium oxalate mixture etc.), lactate buffers (e.g., lactic acid-sodium lactate mixture, lactic acid-sodium hydroxide mixture, lactic acid-potassium lactate mixture etc.) and acetate buffers (e.g., acetic acid-sodium acetate mixture, acetic acid-sodium hydroxide mixture etc.). Phosphate buffers, carbonate buffers, histidine buffers, trimethylamine salts, such as Tris, HEPES and other such known buffers can be used.

Preservatives may be added to the compositions of the present invention to retard microbial growth, and may be added in amounts ranging from 0.2%-1% (w/v). Suitable preservatives for use with the present invention include phenol, benzyl alcohol, m-cresol, octadecyldimethylbenzyl ammonium chloride, benzyaconium halides (e.g., chloride, bromide and iodide), hexamethonium chloride, alkyl parabens, such as, methyl or propyl paraben, catechol, resorcinol, cyclohexanol and 3-pentanol.

Isotonicifiers can be present to ensure physiological isotonicity of liquid compositions of the instant invention and include polhydric sugar alcohols, preferably trihydric or higher sugar alcohols, such as glycerin, erythritol, arabitol, xylitol, sorbitol and mannitol. Polyhydric alcohols can be present in an amount of between about 0.1% to about 25%, by weight, preferably 1% to 5% taking into account the relative amounts of the other ingredients.

Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols; amino acids, such as arginine, lysine, glycine, glutamine, asparagine, histidine, alanine, ornithine, L-leucine, 2-phenylalanine, glutamic acid, threonine etc.; organic sugars or sugar alcohols, such as lactose, trehalose, stachyose, arabitol, erythritol, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, a-monothioglycerol and sodium thiosulfate; low molecular weight polypeptides (i.e., <10 residues); proteins, such as human serum albumin, bovine serum albumin, gelatin or immunoglobulins; hydrophilic polymers, such as polyvinylpyrrolidone, saccharides, monosaccharides, such as xylose, mannose, fructose or glucose; disaccharides, such as lactose, maltose and sucrose; trisaccharides, such as raffinose; polysaccharides, such as, dextran and so on. Stabilizers can be present in the range from 0.1 to 10,000 w/w per part of biopolymer.

Additional miscellaneous excipients include bulking agents, (e.g., starch), chelating agents (e.g., EDTA), antioxidants (e.g., ascorbic acid, methionine or vitamin E) and co-solvents.

Non-ionic surfactants or detergents (also known as “wetting agents”) may be added to help solubilize the therapeutic agent, as well as to protect the therapeutic protein against agitation-induced aggregation, which also permits the formulation to be exposed to shear surface stresses without causing denaturation of the protein. Suitable non-ionic surfactants include polysorbates (20, 80 etc.), polyoxamers (184, 188 etc.), Pluronic® polyols and polyoxyethylene sorbitan monoethers (TWEEN-20®, TWEEN-80® etc.). Non-ionic surfactants may be present in a range of about 0.05 mg/ml to about 1.0 mg/ml, preferably about 0.07 mg/ml to about 0.2 mg/ml.

The instant invention encompasses formulations, such as, liquid formulations having stability at temperatures found in a commercial refrigerator and freezer found in the office of a physician or laboratory, such as from about 20° C. to about 5° C., said stability assessed, for example, by microscopic analysis, for storage purposes, such as for about 60 days, for about 120 days, for about 180 days, for about a year, for about 2 years or more. The liquid formulations of the present invention also exhibit stability, as assessed, for example, by particle analysis, at room temperatures, for at least a few hours, such as one hour, two hours or about three hours prior to use.

Examples of diluents include a phosphate buffered saline, buffer for buffering against gastric acid in the bladder, such as citrate buffer (pH 7.4) containing sucrose, bicarbonate buffer (pH 7.4) alone, or bicarbonate buffer (pH 7.4) containing ascorbic acid, lactose, or aspartame. Examples of carriers include proteins, e.g., as found in skim milk, sugars, e.g., sucrose, or polyvinylpyrrolidone. Typically these carriers would be used at a concentration of about 0.1-90% (w/v) but preferably at a range of 1-10%

The formulations to be used for in vivo administration must be sterile. That can be accomplished, for example, by filtration through sterile filtration membranes. For example, the formulations of the present invention may be sterilized by filtration.

The stimulators of RGS2/4 protein expression can be administered to a patient at therapeutically effective doses to prevent, treat, or control the condition. The compounds are administered to a patient in an amount sufficient to elicit an effective protective or therapeutic response in the patient. An effective therapeutic response is a response that at least partially arrests or slows the symptoms or complications of the disease. An amount adequate to accomplish this is defined as “therapeutically effective dose.” The dose will be determined by the efficacy of the particular RGS2/4 stimulator employed and the condition of the subject, as well as the body weight or surface area of the area to be treated. The size of the dose also will be determined by the existence, nature, and extent of any adverse effects that accompany the administration of a particular compound in a particular subject.

It will be understood by those of ordinary skill in the art, that based on our lab results so far, and in the literature which gave conversion for digoxin 1 ng/ml=1.28 nM, and safety, the compositions and methods of treatment claimed herein, would provide sustained levels of 100 fentograms/ml to 100 picograms/ml of the particular RGS2/4 stimulator in the affected tissue, and no more than 100 pg/ml in plasma to avoid effects upon the heart. It is intended that within this plasma range the range therapeutic for heart arrhythmias (0.5 to 2 ng/ml plasma levels) and the toxic range which is typically greater than 5 ng/ml will be avoided.

It is understood by those of ordinary skill in the art of oral dosing of digoxin for cardiac arrhythmias for children under 2 years of age that the oral dose is 1 mcg/kg/day divided bid or approximately 2.5 mcg twice a day, for older children it is approximately 25 mcg per day and a typical adult dose about 20 mcg per day.

In order to maintain sustained low concentrations of the particular RGS2/4 stimulator, a range of transdermal formulations are contemplated as described herein. In an embodiment, the particular RGS2/4 stimulator would be provided, for example, in a transdermal patch or cream at a dose range approximately equivalent to 1/50th or 1/100th of the oral therapeutic dose, and the absorption would be only local instead of systemic-therefore the systemic levels would be approaching undetectable and therefore would not produce heart toxicity. For example, in a topical preparation of the compositions of the present invention would include a concentration range of a particular RGS2/4 stimulator, and dosing regimen, such as with digoxin, of about 10 mcg once a day, 1 mcg twice a day, 0.1 mcg twice a day, 0.01 mcg twice a day, and 0.001 mcg twice a day (to produce roughly estimated plasma blood levels between 100 pg/ml to 100 fm/ml for infants through adults.

In accordance with one or more embodiments, there is provided ophthalmic formulations comprising stimulators of RGS2 and/or RGS4 include, for example, cardiotonic steroids or cardiac glycosides, wherein the formulation is suitable for administration to the eye of a subject. The ophthalmic formulation may have a pH between 5.5 and 7. In some embodiments the ophthalmic formulation is an aqueous formulation. In some embodiments the ophthalmic formulation is in the form of a single dose unit. In some embodiments the ophthalmic formulation does not comprise a preservative. The ophthalmic formulation may further comprise one or more additional therapeutic agents, such as antioxidants. The ophthalmic formulation may further comprise one or more tear substitutes. In some embodiments, at least one of the tear substitutes contains an ophthalmic lubricant (e.g., hydroxypropylmethylcellulose).

A variety of tear substitutes are known in the art and include, but are not limited to: monomeric polyols, such as, glycerol, propylene glycol, and ethylene glycol; polymeric polyols such as polyethylene glycol; cellulose esters such hydroxypropylmethyl cellulose, carboxy methylcellulose sodium and hydroxy propylcellulose; dextrans such as dextran 70; water soluble proteins such as gelatin; vinyl polymers, such as polyvinyl alcohol, polyvinylpyrrolidone, and povidone; and carbomers, such as carbomer 934P, carbomer 941, carbomer 940 and carbomer 974P. Many such tear substitutes are commercially available, which include, but are not limited to cellulose esters such as Bion Tears®, Celluvisc®, Genteal®, OccuCoat®, Refresh®, Teargen II®, Tears Naturale®, Tears Natural II®, Tears Naturale Free®, and TheraTears®; and polyvinyl alcohols such as Akwa Tears®, HypoTears®, Moisture Eyes®, Murine Lubricating®, and Visine Tears®. Tear substitutes may also be comprised of paraffins, such as the commercially available Lacri-Lube® ointments. Other commercially available ointments that are used as tear substitutes include Lubrifresh PM®, Moisture Eyes PM® and Refresh PM®. Preservatives and other additives may also be present such as, for example, antimicrobials, antioxidants, chelating agents, and inert gases and the like.

Therefore, in accordance with some embodiments, the stimulators of RGS2 and/or RGS4 can be formulated in ophthalmic drops. In an embodiment, such an ophthalmic formulation would include the stimulators of RGS2 and/or RGS4 in a concentration of about 0.001% to 0.0001% w/v to provide the same low dosage range as above in the eye, that is 100 fentograms/ml to 100 picograms/ml.

In accordance with some embodiments, the present invention provides intranasal formulation of the RGS2/4 stimulators. Preferred embodiments of the invention can comprise stable aqueous solutions of RGS2/4 stimulators, such as digoxin, or one or more of its salts, which can be used in the form of inhalation solution, pressurized aerosol, eye drops or nasal drops, and in a particular preferred embodiment, in the form of a spray (preferably a nasal spray). The spray can, for example, be formed by the use of a conventional spray-squeeze bottle or a pump vaporizer. In addition, it is also possible to use compressed gas aerosols. In a preferred embodiment, 0.001% to 0.0001% w/v of RGS2/4 stimulators should be released per individual actuation.

The formulations preferably contain a preservative and/or stabilizer. These include, for example: ethylene diamine tetra-acetic acid (edetic acid) and its alkali salts (for example dialkali salts such as disodium salt, calcium salt, calcium-sodium salt), lower alkyl p-hydroxybenzoates, chlorhexidine (for example in the form of the acetate or gluconate) and phenyl mercury borate. Other suitable preservatives are: pharmaceutically useful quaternary ammonium compounds, for example cetylpyridinium chloride, tetradecyltrimethyl ammonium bromide, generally known as “cetrimide”, benzyldimethyl-[2-[2-[p-(1,1,3,3-tetramethyl-butyl)phenoxy]ethoxy]-ammonium chloride, generally known as “benzethonium chloride” and myristyl picolinium chloride. Each of these compounds may be used in a concentration of 0.002 to 0.05%, for example 0.02% (weight/volume in liquid formulations, otherwise weight/weight). Preferred preservatives among the quaternary ammonium compounds are, however, alkylbenzyl dimethyl ammonium chloride and mixtures thereof, for example the compounds generally known as “benzalkonium chloride.”

The total amount of preservatives in the formulations (solutions, ointments, etc.) is preferably from 0.001 to 0.10 g, preferably 0.01 g per 100 ml of solution/suspension or 100 g of formulation.

In the case of preservatives, the following amounts of individual substances can, for example, be used: thimerosal 0.002-0.02%; benzalkonium chloride 0.002 to 0.02% (in combination with thimerosal the amount of thimerosal is, for example=0.002 to 0.005%); chlorhexidine acetate or gluconate 0.01 to 0.02%; phenyl mercuric/nitrate, borate, acetate 0.002-0.004%; p-hydroxybenzoic acid ester (for example, a mixture of the methyl ester and propyl ester in the ratio 7:3): preferably 0.05-0.15, more preferably 0.1%.

The preservative used is preferably a combination of edetic acid (for example, as the disodium salt) and benzalkonium chloride. In this combination, the edetic acid is preferably used in a concentration of 0.05 to 0.1%, benzalkonium chloride preferably being used in a concentration of 0.005 to 0.05%, more preferably 0.01%.

In the case of solutions/suspensions reference is always made to percent by weight/volume, in the case of solid or semi-solid formulations to percent by weight/weight of the formulation.

Further auxiliary substances which may, for example, be used for the formulations of the invention are: polyvinyl pyrrolidone, sorbitan fatty acid esters such as sorbitan trioleate, polyethoxylated sorbitan fatty acid esters (for example polyethoxylated sorbitan trioleate), sorbimacrogol oleate, synthetic amphotensides (tritons), ethylene oxide ethers of octylphenolformaldehyde condensation products, phosphatides such as lecithin, polyethoxylated fats, polyethoxylated oleotriglycerides and polyethoxylated fatty alcohols. In this context, polyethoxylated means that the relevant substances contain polyoxyethylene chains, the degree of polymerisation of which is generally between 2 to 40, in particular between 10 to 20. These substances are preferably used to improve the solubility of the azelastine component.

It is optionally possible to use additional isotonization agents. Isotonization agents which may, for example, be used are: saccharose, glucose, glycerine, sorbitol, 1,2-propylene glycol and NaCl.

The isotonization agents adjust the osmotic pressure of the formulations to the same osmotic pressure as nasal secretion. For this purpose, these substances are in each case to be used in such amount that, for example, in the case of a solution, a reduction in the freezing point of 0.50 to 0.56° C. is attained in comparison to pure water.

The toxicity and therapeutic efficacy of such compounds and compositions of the present invention can be determined by standard pharmaceutical procedures in cell cultures or experimental animals, for example, by determining the LD₅₀ (the dose lethal to 50% of the population) and the ED₅₀ (the dose therapeutically effective in 50% of the population). The dose ratio between toxic and therapeutic effects is the therapeutic index and can be expressed as the ratio, LD₅₀/ED₅₀. Compounds that exhibit large therapeutic indices are preferred. While compounds that exhibit toxic side effects can be used, care should be taken to design a delivery system that targets such compounds to the site of affected tissue to minimize potential damage to normal cells and, thereby, reduce side effects.

The data obtained from cell culture assays and animal studies of the present invention can be used to formulate a dosage range for use in humans. The dosage of such compounds lies preferably within a range of circulating concentrations that include the ED₅₀ with little or no toxicity. The dosage can vary within this range depending upon the dosage form employed and the route of administration. For any compound used in the methods of the invention, the therapeutically effective dose can be estimated initially from cell culture assays. A dose can be formulated in animal models to achieve a circulating plasma concentration range that includes the IC₅₀ (the concentration of the test compound that achieves a half-maximal inhibition of symptoms) as determined in cell culture. Such information can be used to more accurately determine useful doses in humans. Levels in plasma can be measured, for example, by high performance liquid chromatography (HPLC). In general, the dose equivalent of a modulator is from about 1 ng/kg to 10 mg/kg for a typical subject.

The present invention also provides kits for compound screening or therapeutic applications. For compound screening applications, such kits may include any or all of the following: assay reagents, buffers, RGS2/4 expressing cells, GNAQ mutant containing cells, primers, antibodies, or the like.

In addition, the kits may include instructional materials containing directions (i.e., protocols) for the practice of the methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### Examples

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

Testing of digoxin and ouabain for blocking of overactivation of ERK-phosphorylation.

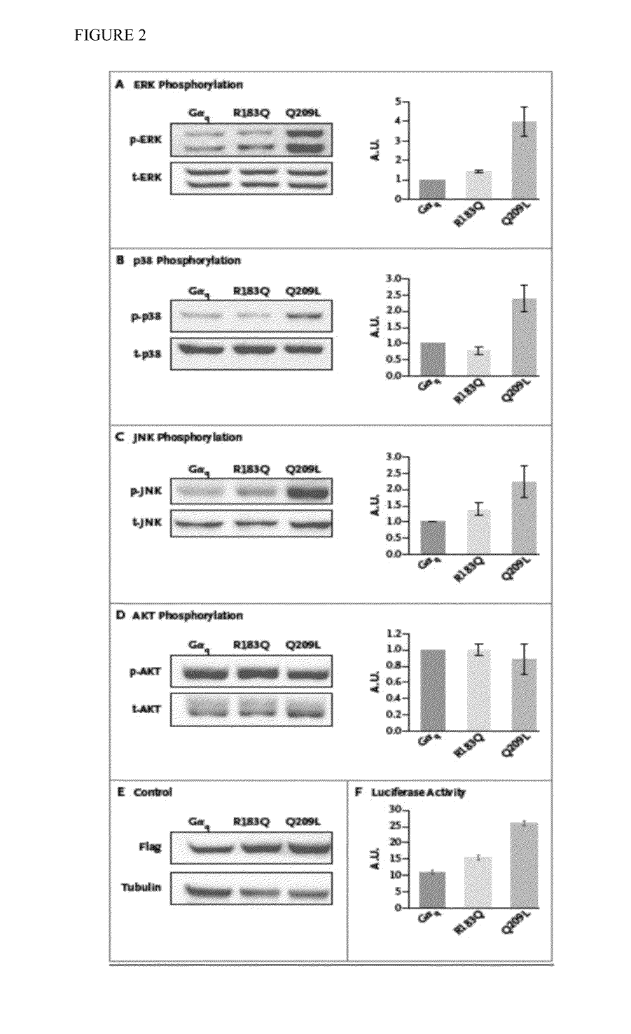

293T cells will be transiently transfected with wildtype, GNAQ, the p.Arg183Gln, or the p.Gln209Leu mutant constructs. Fugene6 (Promega) is used for the 293T transfections (3:1 ratio Fugene6:DNA). The cells are transfected at 60-75% confluency for about an hour. Transiently transfected cells with these mutant constructs have been shown to demonstrate constitutive hyperphosphorylation of ERK and JNK (FIG. 2) (figure from U.S. Provisional Application No. 61/812,309).

Cells will be treated with either digoxin (dose range of 100 fm-100 nM) or ouabain (dose range of 100 fm-100 nM.) or vehicle in vitro for 24 hours in regular media and then the cells lysed and the protein isolated and quantified using Micro BCA Protein Assay Kit (PIERCE 23235).

Levels of phosphorylated-ERK (rabbit monoclonal Phospho-p44/42 MAPK (Erk1/2) Cell Signaling Technology) and phosphorylated-JNK are measured by western analysis and normalized to tubulin. RGS2 and/or RGS4 levels are measured by western analysis and normalized to tubulin. Protein samples are collected from three replicates per cell type and condition. Additionally, similar experiments are performed with the p.Gln209Leu GNAQ mutants as well.

In an embodiment, cells were transfected with p.R183Q and treated with digoxin (10 or 100 nM dose) (Sigma Aldrich D6770 reconstituted in DMEM and 0.04% DMSO) or vehicle (DMEM and 0.04% DMSO) 24 hours post transfection. Protein was isolated 18 hours and 42 hours (n=3-4 protein samples for each treatment group and timepoint. Cells for 42 hour group were split, re-plated, and re-transfected at 18 hours. Drug or vehicle treatment was continued throughout. In addition, HEK 293T cells without transfection were treated with 1, 10, or 100 nM digoxin versus drug. Myc tag was checked by westerns and was present and demonstrated good transfections (data not shown).

100 nM Digoxin for 18 hr was first attempted as these were the treatment conditions that were effective as reported in the Sjögren et al. paper Molecular Pharm 2012. However in the cells transfected for 18 hours with the R183Q mutation 100 nM digoxin treatment p-ERK went up significantly (ratio=3 fold increase, p<0.001). RGS2 ratio was not significantly changed (ratio 1.2, p=0.4) with increased cell death noted in the drug treated cell cultures.

The period of drug exposure was extended because the effect of digoxin may require sufficient time for increasing in RGS2 protein to occur in the cell through decreased protein degradation. In the R183Q mutation cell cultures treated for 42 hours in either 100 nM digoxin or vehicle, RGS2 was increased significantly (ratio 1.7, p=0.047) but p-ERK was also significantly increased about 3.9 fold, p=0.0003 (data not shown). There increased cell death/less growth in the drug treated cells and the drug treated cells looked less healthy and less branched.

To minimize cell death with the drug treated cultures the dose was decreased to 10 nM digoxin. In cells treated with digoxin for 42 hours, RGS2 was increased in the drug treated cells compared to the vehicle (ratio=1.7, p=0.07). p-ERK went up only mildly (ratio 1.3, p=0.01) at 42 hours. Untransfected cells that received 100 nM digoxin for 18 hours (n=1 vehicle and n=1 digoxin) showed an increase in RGS2 (ratio 1.6) and a decrease in p-ERK (ratio 0.6 respectively) (data not shown).

The results show that digoxin or ouabain treatment will increase protein levels of RGS2 and/or RGS4 in the mutated cells and correct the constitutive hyperphosphorylation of ERK that have already been noted in these cells.

Digoxin doses ranging from 100 fM to 1 nM over a 42 hour duration will be tested to determine if the hypothesis is correct that the cells with the R183 mutation have increased sensitivity to the drug and therefore must be treated with lower doses over a longer period of time. Once this is completed we will work with non-transfected/WT transfected cells at the 100 nM dose and the doses that are most effective in increasing RGS2 and decreasing p-ERK in the R183 cells.

Digoxin Topical Formulation.

Digoxin is known to be absorbed through the skin. This has already been studied in and reported (Altern Lab Anim. 2008 May; 36(2):161-87). Topical formulations for dermal applications of steroids are well known in the art. In an embodiment, the present invention provides a pharmaceutical composition for topical application comprising a topically active cardiotonic steroid in a multiple emulsion in which the continuous phase is aqueous and the primary disperse phase is an oil phase in which a hydrophilic phase containing the steroid in saturated solution is dispersed.

For a solvent phase forming material, any good solvent which is not miscible with the oil phase can be used for the steroid in question. Preferably glycols, e.g. propylene glycol, butandiols, polyethylene glycols are used. The amount of these solvent phase forming materials depends on the solubility properties of the steroid, but is usually 0.2-5% (w/w) of the complete composition.

For the oil phase forming material, a fat with suitable viscosity range is used, for example petrolatum or mixtures of petrolatum and animal oils, such as wool grease, mixtures of petrolatum and vegetable oils, such as peanut oil, olive oil, mixtures of petrolatum and mineral oils, such as paraffin oil, isopropylmyristate, isopropylpalmitate and triglycerides, mixtures of petrolatum and synthetic oils, such as silicone oils. The amount of these oil phase forming materials depends on the desired fat content of the multiple emulsion but is generally in the range 10-50% (w/w) of the multiple emulsion.

The continuous phase can consist of 40-80% (w/w) of water, and a water-soluble emulsifier. This phase may also consist of a fatty alcohol, such as cetostearyl alcohol. The emulsifier to be used for the formation of the dispersion of the solution of steroid in the oil phase should have such an appropriate hydrophilic-lipophilic balance that the oil phase used can form the dispersion medium of the solution in the oil emulsion. Beeswax, sorbitan fatty esters and monoglycerides are preferably used as emulsifiers. The amount of oil-soluble emulsifier is usually 1-20% (w/w) of the amount of the oil phase.

The water soluble emulsifier is an emulsifier having a hydrophilic-lipophilic balance at which the oil phase can form the dispersed phase of the oil in water emulsion. Preferably a non-ionic emulsifier, for example cetomacrogol 1000 [CH₃(CH₂)ₘO(CH₂OCH₂)ₙ—CH₂ OH where m may be 15 or 17 and n may be 19 to 23], is used. The amount of the water-soluble emulsifier to be added is typically in the range 1-20% (w/w) of the amount of the oil phase described above.

The preparation of the topical steroid composition starts with dissolving the steroid in the most suitable solvent. The steroid solution in oil emulsion is then prepared from the oil phase forming material and the steroid solution. The oil-soluble emulsifier and the steroid solution is added to the oil phase forming material which is heated and agitated to form a steroid solution in oil emulsion. Then this steroid solution in oil emulsion is added to an aqueous solution prepared by dissolving the water-soluble emulsifier in water to form the multiple emulsions during heating and agitation.

For example, the topical cardiotonic steroid formulation of the present invention can include, in an embodiment, the internal steroid solution phase: digoxin 0.025 gram, propylene glycol 2.5 gram (II), oil phase petrolatum 21.25 gram bees wax 1.25 gram (III), external water phase is cetostearyl alcohol 3.0 gram, cetomacrogol 1000 2.0 gram, and distilled water 70.0 gram.

The digoxin is dissolved in propylene glycol during heating to 70° C. making up solution (I). Solution (I) is added to solution (II) at 70° C., and the mixture is subjected to agitation by a homomixer to prepare a steroid solution in oil emulsion. This emulsion is added to (III), preheating to 70° C., during agitation by a homomixer, and results in the multiple emulsions. After cooling to room temperature, during gentle agitation, a soft white shiny cream is obtained.

Phase 1 Clinical Trial: Study safety, dose range and identify side effects.

With IRB approval, a small number of subjects are recruited (n=20) for application of a topical formulation of digoxin (cream or gel) on a small area of port-wine stain birthmark skin daily over a period of weeks. Informed consent is obtained from every study subject, and all treatment and sample collection is performed under the auspices of Johns Hopkins Medicine Institutional Review Board-approved protocols. All patient samples are de-identified, with clinical and laboratory features linked only to the patient code. A range of dose concentrations (0.01-2%) is applied over the period of the study and any sign of skin irritation or adverse effect carefully noted and used to adjust dose range in this initial phase 1 study. The size, color, response, and texture of the port-wine stain birthmark is carefully recorded with photographs, measurements and a birthmark severity scale currently under development.

Phase 2 Clinical Trial: Establishment of the testing protocol to further establish safety and assess effectiveness.

After the Phase 1 Trial, if results are successful, then plans are made to compare drug to topically applied vehicle in a Phase 2 clinical trial design. The trial is randomized and placebo controlled. With IRB approval, subjects would be recruited (n=100) and randomized to receive either application of a topical formulation of digoxin (cream or gel) or vehicle without active drug. Study drug or placebo is applied to a small area of port-wine stain birthmark skin daily over a period of weeks, using the dose(s) identified in the phase 1 clinical trial to be both safe and effective. Any sign of skin irritation or adverse effect carefully noted and used to adjust dose range. The size, color, response, and texture of the port-wine stain birthmark is carefully recorded with photographs, measurements and a birthmark severity scale currently under development.

Phase 3 Clinical Trial: Establishing Effectiveness and confirming safety.

Depending on the results of the Phase 2 clinical trial the advisability and need for proceeding to a Phase 3 Clinical trial would be determined and the protocol for the randomized placebo controlled study in a larger group of subjects determined after a power analysis is completed and will be based on the results of the Phase 2 Clinical Trial.

Screening Assay.

In accordance with some embodiments, the present invention provides a screening assay that is useful for identifying molecules that enhance RGS2 and/or RGS4 protein levels and function in vitro. The methods are based on a known method (Mol. Pharm., 82:500-509 (2012)) to identify digoxin and ouabain as drugs which enhance RGS2 and/or RGS4 protein levels and function. As described herein, T293 cells will be transiently or stably transfected with human RGS2-PL or RGS4-PL which enables measurement of RGS2 and/or RGS4 levels by chemiluminescence. In a preferred embodiment, the cells will be stably transfected with human RGS2-PL or RGS4-PL as described below. These cells will then be used to screen a large library of compounds and to identify other drugs which increase levels of RGS2 and/or RGS4.

Mammalian Expression Constructs.

A 4-kDa fragment of β-galactosidase [ProLabel (PL); DiscoveRx, Freemont, CA] is added to the C terminus of human RGS2 and RGS4 (i.e., RGS2-PL and RGS4-PL). Human RGS2 and RGS4 are amplified through PCR from the pcDNA3.1 RGS2-HA and RGS4-HA vectors described previously (Mol. Pharmacol., 71:1040-1050 (2007)). Full-length RGS open reading frames, without the HA tag, are cloned into the pCMV-ProLabel-C3 vector (DiscoveRx) in-frame with the C-terminal ProLabel tag. Restriction sites for XhoI (5′) and BamHI (3′) are added to the RGS2 and RGS4 PCR primers to facilitate insertion into the pCMV-ProLabel-C3 vector. The primers for amplification of RGS2 are 5′-CGCTCGAGATGCAAAGTGCTATGTTCTTGGC-3′ (sense) (SEQ ID NO: 1) and 5′-CCGCTCGAGATGCAAAGTGCTATGTTCTTGGC-3′ (antisense) (SEQ ID NO: 2). The primers for amplification of RGS4 are 5′-CCGCTCGAGATGTGCAAAGGGCTTGCAGGTCTGCC-3′ (sense) (SEQ ID NO: 3) and 5′-CGCGGATCCGGCACACTGAGGGACCAGGG-3′ (antisense) (SEQ ID NO: 4).

Cell Culture and Transfections.

Human embryonic kidney (HEK) 293 or HEK-293T cells are maintained in a humidified incubator at 37° C. with 5% CO, and are grown to 95% confluence in Dulbecco's modified Eagle's medium (DMEM; Invitrogen) containing 4.5 g/liter glucose, 2 mM/L-glutamine, 25 mM HEPES, 10% fetal bovine serum (FBS), 100 U/ml penicillin, and 100 μg/ml streptomycin. Cells are transfected by using Lipofectamine 2000 (Invitrogen) at 5 μl/μg of plasmid DNA, according to the manufacturer's recommended protocol. All transfections are performed under serum-free conditions in Opti-MEM (Invitrogen). Transfections are allowed to proceed for 4 to 5 hours before the medium was changed to DMEM with 10% FBS. For transient transfections, experiments are performed 48 hours after transfection.

To develop stable HEK-293 cell lines expressing RGS2-PL and/or RGS4-PL, 400 μg/ml G-418 (Geneticin; Invitrogen) is added to the cells 48 hours after transfection. The cells are cultured until nontransfected cells died. For selection of individual clones, cells are sorted into 96-well plates (at 1 cell/well) through flow cytometry, and cells are allowed to grow in selection medium until colonies formed. Individual clones are expanded to 12-well plates.

PathHunter ProLabel Galactosidase Complementation Assay.

The 4-kDa PL tag on the C terminus of RGS2 and/or RGS4 permits rapid quantitative assessment of protein expression. HEK-293 cells expressing RGS2-PL and/or RGS4-PL are trypsinized and resuspended in DMEM without phenol red (Invitrogen) containing 4.5 g/liter glucose, 2 mM L-glutamine, 25 mM HEPES, and 0.1% bovine serum albumin and are counted by using a cell counter. Cells are diluted to 5×10⁵ cells/ml and are plated at 15×10³ cells/well in a white 384-well plate (Corning Life Sciences, Lowell, Mass.), in 30 μl of DMEM without phenol red containing 0.1% bovine serum albumin. Cells are allowed to attach for at least 3 hours before treatment with compounds (1-100 μM) At the end of treatment, the medium is removed and CellTiter-Fluor viability reagent (5 μl/well; Promega, Madison, Wis.) is added. The plate is shaken at 400 rpm for 2 minutes and incubated at 37° C. for 30 minutes before fluorescence (excitation, 390 nm; emission, 505 nm) was measured.

Development of Novel Compounds Identified by Screening Assay. In vitro testing of novel compounds. Novel compounds or pharmaceutical compositions identified by the screening assay above that significantly increase RGS levels, are then tested in vitro in the transiently transfected cells with the GNAQ p.Arg183Gln or with the p.Gln209Leu amino acid substitution as described above for digoxin. Stable cell lines with the p.Arg183Gln and with the p.Gln209Leu GNAQ mutants are currently developing for this use. If successful then these stable cells lines can be used instead of transiently transfected cells to demonstrate effectiveness of drugs in normalizing hyperactivation of downstream (ERK-p and JNK-p) pathways. Possible libraries to be screened using this assay methods include the Eisai compound library through the JHU, Eisai drug-discovery research collaboration for brain disorders, and other libraries available through pharmaceutical companies.

Preclincial Animal testing. Novel drug candidates or formulations which are positive in in vitro testing then move to pilot preclinical testing in animals for safety and pharmacologic studies. Pharmacological testing would begin in adult and immature rats to determine GI absorption, tolerability and half-life in healthy animals. Efforts will be underway soon to develop an animal model of SWS. When available, this model will be used to evaluate system administration of the drug for effectiveness and safety in ameliorating the manifestations of the condition compared to vehicle with a range of doses used.

Clinical testing. Promising novel compounds or pharmaceutical compositions that are effective in preclinical models would move to pilot clinical testing. Initially in the skin of normal subjects and subjects and subjects with port-wine stain birthmarks. The same approach used to evaluate the clinical effectiveness of digoxin outlined in point number 3 above would be used to evaluate additional candidates. If warranted, systemic safety testing in normal subjects and subjects with Sturge-Weber syndrome. This would take a similar approach except that the outcomes monitored for assessment of effectiveness would be different from that in testing topical agents and could include impact on seizures, neurologic status, hemiparesis and visual field cut as quantified by SWS neuroscore as well as by medical rehabilitation scales, neuroimaging, quantitative EEG, transcraniel Doppler and urine angiogenesis biomarkers.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for treating Sturge-Weber Syndrome, Klippel-Trenaunay-Weber Syndrome, Port-Wine Stains and related neurocutaneous disorders in a subject comprising administering to the subject a therapeutically effective amount of a pharmaceutically acceptable composition which increases RGS2 and/or RGS4 protein levels in the subject.

2. The method of claim 1, wherein the composition comprises at least one cardiotonic steroid.

3. The method of claim 2, wherein the at least one cardiotonic steroid is selected from the group consisting of digitalis, digoxin, ouabain, deslanoside, lanatoside C, acetyldigoxin, G-strophanthin, and derivatives thereof.

4. The method of claim 3, wherein the composition is administered to the subject topically, subcutaneously, intravenously, ocularly, intranasally, or orally.

5. The method of claim 4, wherein the composition is administered to the subject topically.

6. The method of claim 1, wherein the pharmaceutical composition comprises at least one other active agent.

7. A method for identifying a molecule which increases RGS2 and/or RGS4 protein levels in a cell or population of cells having the GNAQ Arg183Gln amino acid substitution comprising:
a) obtaining a cell or population of cells which express GNAQ having the Arg183Gln amino acid substitution and a cell or population of cells having wild type GNAQ;
b) incubating the molecule with both cells or population of cells of a);
c) measuring the levels of RGS2 and or RGS4, in both cells or population of cells of a);
d) comparing the levels of RGS2 and or RGS4 in both cells or population of cells of a); and
e) determining that the molecule increases RGS2 and/or RGS4 protein levels in a cell or population of cells having the GNAQ Arg183Gln amino acid substitution when the protein levels are greater than in the cell or population of cells having wild type GNAQ.

8. The method of claim 7, further comprising:
c1) measuring the levels of phosphorylated ERK in both cells or population of cells of a);
d1) comparing the levels of phosphorylated ERK in both cells or population of cells of a); and
e1) determining that the molecule decreases phosphorylated ERK levels in a cell or population of cells having the GNAQ Arg183Gln amino acid substitution when the phosphorylated ERK levels are less than in the cell or population of cells having wild type GNAQ.

9. The method of claim 8, further comprising:
c2) measuring the levels of phosphorylated JNK in both cells or population of cells of a);
d2) comparing the levels of phosphorylated JNK in both cells or population of cells of a); and
e2) determining that the molecule decreases phosphorylated JNK levels in a cell or population of cells having the GNAQ Arg183Gln amino acid substitution when the phosphorylated JNK levels are less than in the cell or population of cells having wild type GNAQ.

10. The method of claim 9, wherein the molecule is a peptide, protein, siRNA, antibody, small molecule or cardiotonic steroid.

11. A method for identifying a molecule which increases RGS2 and/or RGS4 protein levels in a cell or population of cells having the GNAQ Arg183Gln amino acid substitution comprising:
a) obtaining a cell or population of cells which express RGS2 and/or RGS4 protein which are capable of emitting a photon when stimulated;
b) incubating the molecule with the cell or population of cells of a);
c) measuring the levels of photo emission, in the cell or population of cells of a);
d) comparing the levels of RGS2 and or RGS4 in the cell or population of cells of a) to that of a control; and
e) determining that the molecule increases RGS2 and/or RGS4 protein levels in a cell or population of cells when the protein levels are greater than the control.

12. A method for treatment of a GNAQ dependent melanoma in a subject comprising administering to the subject a therapeutically effective amount of a pharmaceutically acceptable composition which increases RGS2 and/or RGS4 protein levels in the subject.

13. The method of claim 12, wherein the composition comprises at least one cardiotonic steroid.

14. The method of claim 13, wherein the at least one cardiotonic steroid is selected from the group consisting of digitalis, digoxin, ouabain, deslanoside, lanatoside C, acetyldigoxin, G-strophanthin, and derivatives thereof.

15. The method of claim 14, wherein the composition is administered to the subject topically, subcutaneously, intravenously, ocularly, intranasally, or orally.

16. The method of claim 15, wherein the composition is administered to the subject ocularly.

17. The method of claim 16, wherein the pharmaceutical composition comprises at least one other active agent.
